(19) United States
(12) Patent Application Publication
(10) Pub. No.: US 2025/0170877 A1
(43) Pub. Date: May 29, 2025

(54) VEHICLE INTERIOR STERILIZER

(71) Applicants: HYUNDAI MOTOR COMPANY, Seoul (KR); KIA CORPORATION, Seoul (KR); SL CORPORATION, Daegu (KR)

(72) Inventors: Jae Seung Lee, Hwaseong-si (KR); Hyun Woo Choi, Daegu (KR); Kwang Soo Park, Daegu (KR); Myeong Joo Seo, Gyeongsan-si (KR); Ji Hoon Yang, Gyeongsan-si (KR); Young Eon Jung, Daegu (KR); Hee Kwang Park, Gyeongsan-si (KR)

(73) Assignees: HYUNDAI MOTOR COMPANY, Seoul (KR); KIA CORPORATION, Seoul (KR); SL CORPORATION, Daegu (KR)

(21) Appl. No.: 18/652,468

(22) Filed: May 1, 2024

(30) Foreign Application Priority Data
Nov. 23, 2023 (KR) 10-2023-0164018

(51) Int. Cl. B60H 3/00; A61L 2/10; B60J 7/00

(52) U.S. Cl. CPC B60H 3/0078; A61L 2/10; B60J 7/0007; A61L 2202/16

(57) ABSTRACT

A vehicle interior sterilizer includes: a roof lining and a housing inserted into and located in the roof lining. The sterilizer has a cover unit that can open and close an opening of the housing. The sterilizer has an actuator unit coupled to a shaft of the cover unit and configured to apply a driving force to selectively open and close the cover unit. The sterilizer has a light source unit located in the opening of the housing and exposed to an interior of a vehicle to perform sterilization when the cover unit is opened. The sterilizer has a controller configured to determine whether an occupant has exited the vehicle and to open the cover unit and turn on the light source unit by driving of the actuator unit.

### CROSS-REFERENCE TO RELATED APPLICATION

This application claims, under 35 U.S.C. § 119 (a), the benefit of and priority to Korean Patent Application No. 10-2023-0164018, filed on Nov. 23, 2023, the entire contents of which are incorporated herein by reference.

### BACKGROUND

### (a) Technical Field

The present disclosure relates to a vehicle interior sterilizer. More particularly, the present disclosure relates to a vehicle interior sterilizer capable of sterilizing a vehicle interior through a light source unit by selectively opening a cover unit of the sterilizer.

### (b) Background Art

In order to keep an air environment in the vehicle comfortable or suitable for a driver, a vehicle air conditioner is provided. The vehicle air conditioner has various functions such as heating and cooling, which are the main functions in the vehicle. Other functions include dehumidification, humidification, purification, ventilation, aromatization, and functions of constantly maintaining temperature and humidity.

In the vehicle air conditioner, air flows into the interior of the vehicle through a cabin filter that filters pollutants such as exhaust gas, dust, and pollen in the air to purify the air. Accordingly, the cabin filter plays an important role in maintaining comfortable interior vehicle air.

The cabin filter is usually located in a glove box in front of the passenger seat. In this case, when the cabin filter is checked or replaced, the glove box should be opened, removed, and reinserted. Further, the cabin filter is required to be mounted in consideration of the direction in which air flows, so it is very inconvenient and laborious for a driver to replace the cabin filter frequently. In addition, since it is difficult to immediately check the contamination status of the cabin filter, it takes a considerable amount of time. As a result, it is not easy to replace the cabin filter.

Furthermore, a problem arises in that it is difficult to sterilize bacteria contaminating the vehicle interior surfaces using conventional air conditioners having a function of purifying the air.

The above information disclosed in this Background section is only to enhance understanding of the background of the disclosure. Therefore, the Background section may contain information that does not form the prior art that is already known to a person of ordinary skill in the art.

### SUMMARY

The present disclosure has been made in an effort to solve the above-described problems associated with the prior art. It is an object of the present disclosure to provide a vehicle interior sterilizer located so as not to be exposed to the inside of a roof lining or vehicle headliner.

In addition, the present disclosure provides a vehicle interior sterilizer including a cover unit capable of being selectively opened and closed. The vehicle interior sterilizer may perform sterilization by determining the presence or absence of an occupant in the vehicle.

The objects of the present disclosure are not limited to the above-mentioned objects. Other technical objects not mentioned herein should be more clearly understood by those having ordinary skill in the art to which the present disclosure pertains from the detailed description of the embodiments. Additionally, the objects of the present disclosure may be achieved by the various combinations thereof as indicated in the claims.

In one aspect, the present disclosure provides a vehicle interior sterilizer including a roof lining and a housing inserted into and located in the roof lining. The housing has an opening therein. The sterilizer also includes a cover unit configured to open and close the opening of the housing. The sterilizer also includes an actuator unit coupled to a shaft of the cover unit and configured to apply a driving force to selectively open and close the cover unit. The sterilizer also includes a light source unit located in the opening of the housing and exposed to an interior of a vehicle to perform sterilization when the cover unit is opened. The sterilizer also includes a controller configured to determine whether an occupant exits the vehicle and to open the cover unit and turn on the light source unit by driving of the actuator unit.

In an embodiment, the vehicle interior sterilizer may further include a reflector located adjacent to the light source unit.

In another embodiment, the vehicle interior sterilizer may further include an indicator located in at least one or more of the cover unit, a cockpit module, a console, an instrument cluster monitor, a windshield, or vehicle side glass. The indicator may be configured to light up when the light source unit is driven.

In still another embodiment, the indicator may include a luminescent pigment applied thereto.

In yet another embodiment, the actuator unit may include: a motor configured to provide a driving force; a first worm gear engaged with a central shaft of the motor; a worm wheel gear engaged with the first worm gear; and a second worm gear located between the worm wheel gear and a clutch unit circumscribing the shaft.

In still yet another embodiment, the clutch unit may further include a clutch plate integrally rotated with the shaft and a clutch gear configured to surround an outer surface of the clutch plate and engage with the second worm gear.

In a further embodiment, the vehicle interior sterilizer may further include a spring part configured to surround an outer peripheral surface of the shaft. The spring part may be configured to press the clutch plate so that the clutch plate and the shaft are integrally secured. The sterilizer may also include a stopper disposed to face an end of the spring part and configured to regulate a position of the spring part.

In another further embodiment, the vehicle interior sterilizer may further include a groove formed in the clutch plate and a coupling part located at one end of the shaft. The one end may face the clutch plate, and may be inserted into the groove.

In still another further embodiment, the clutch plate may be configured to prevent rotational force of the cover unit from being applied to the clutch gear when the cover unit is rotated in a direction of closing the cover unit in response to a manual operation.

In yet another further embodiment, the cover unit may be formed of a plurality of plates and the plurality of plates may be configured to move to a side surface of the housing in response to the driving of the actuator unit so as to open the opening of the housing.

In still yet another further embodiment, the controller may be configured to apply power to the actuator unit and to switch the cover unit to an opened state thereof when a sterilization request from a user is approved.

In a still further embodiment, the vehicle interior sterilizer may further include a heat sink located on one side of the light source unit and a cooling fan located on one side of the heat sink.

In another aspect, the present disclosure provides a vehicle interior sterilizer including: a roof lining and a housing inserted into and located in the roof lining. The housing has an opening therein. The sterilizer also includes a cover unit configured to open and close the opening of the housing. The sterilizer also includes an actuator unit coupled to a shaft of the cover unit and configured to apply a driving force so as to selectively open and close the cover unit. The sterilizer may also include a light source unit located in the cover unit and exposed to an interior of a vehicle to perform sterilization when the cover unit is opened. The sterilizer also includes a controller configured to determine whether an occupant exits the vehicle and to open the cover unit and turn on the light source unit by driving of the actuator unit.

In an embodiment, the controller may be configured to control rotational force of the actuator unit so that the cover unit is rotated around the shaft by a predetermined angle when switching the light source unit to an on state thereof.

In another embodiment, the vehicle interior sterilizer may further include an indicator located in at least one or more of the cover unit, a cockpit module, a console, an instrument cluster monitor, a windshield, or vehicle side glass. The indicator may be configured to light up when the light source unit is driven.

Other aspects and embodiments of the disclosure are discussed below.

It should be understood that the terms “vehicle,” “vehicular,” and other similar terms as used herein are inclusive of motor vehicles in general. Such motor vehicle may include sport utility vehicles (SUV), buses, trucks, various commercial vehicles, watercraft including a variety of boats and ships, aircraft, and the like. Such motor vehicles may also include hybrid vehicles, electric vehicles, plug-in hybrid electric vehicles, hydrogen-powered vehicles, and other alternative fuel vehicles (e.g., fuels derived from resources other than petroleum). As referred to herein, a hybrid vehicle is a vehicle that has two or more sources of power, e.g., a vehicle powered by both gasoline and electricity.

The above and other features of the disclosure are discussed below.

### BRIEF DESCRIPTION OF THE DRAWINGS

The above and other features of the present disclosure are described in detail with reference to certain embodiments thereof illustrated in the accompanying drawings, which are given hereinbelow by way of illustration only, and thus are not limitative of the present disclosure, and wherein:

FIG. 1 is a vehicle interior sterilizer located in a roof lining according to an embodiment of the present disclosure;

FIG. 2 is a perspective view of a vehicle interior sterilizer according to an embodiment of the present disclosure;

FIG. 3 is a side cross-sectional view of a vehicle interior sterilizer according to an embodiment of the present disclosure;

FIG. 4 is a rear view of a light source unit of a vehicle interior sterilizer according to an embodiment of the present disclosure;

FIG. 5A is a diagram showing a rotational structure of a cover unit of a vehicle interior sterilizer according to an embodiment of the present disclosure;

FIG. 5B is a diagram showing a coupling structure between a cover unit and an actuator unit of a vehicle interior sterilizer according to an embodiment of the present disclosure;

FIG. 6A is a diagram showing a configuration of a vehicle interior sterilizer according to an embodiment of the present disclosure;

FIG. 6B is a diagram showing an operation of a cover unit of a vehicle interior sterilizer according to an embodiment of the present disclosure;

FIG. 7 is a diagram showing a configuration of a vehicle interior sterilizer according to an embodiment of the present disclosure;

FIG. 8 is a diagram showing an indicator configuration of a vehicle interior sterilizer according to an embodiment of the present disclosure; and

FIG. 9 is a diagram showing a control step of performing sterilization by a controller of a vehicle interior sterilizer according to an embodiment of the present disclosure.

It should be understood that the appended drawings are not necessarily drawn to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

In the figures, reference numbers refer to the same or equivalent parts of the present disclosure throughout the several figures of the drawing.

### DETAILED DESCRIPTION

Hereinafter, reference is made in detail to various embodiments of the present disclosure, examples of which are illustrated in the accompanying drawings and described below. While the technical concept of the disclosure is described in conjunction with the embodiments, it should be understood that the present description is not intended to limit the disclosure to the embodiments. On the contrary, the disclosure is intended to cover not only the disclosed embodiments, but also various alternatives, modifications, equivalents, and other embodiments, which may be included within the spirit and scope of the disclosure as defined by the appended claims. The present embodiments are provided to more fully explain the disclosure to those having ordinary skill in the art.

Terms such as “part,” “controller,” “unit,” and “module” described in the specification mean a unit configured to process at least one function or operation. The unit may be implemented by hardware or software or a combination of hardware and software.

The terms used in the present application are used only to describe specific embodiments and are not intended to limit the present disclosure. Singular forms are intended to include plural forms as well, unless the context clearly indicates otherwise.

In the specification, terms such as “first” and “second” are used to describe various components having the same names, and the terms are used only for the purpose of distinguishing one component from other components. The components are not limited by the terms in the following description.

When a controller, component, device, element, part, unit, module, or the like of the present disclosure is described as having a purpose or performing an operation, function, or the like, the controller, component, device, element, part, unit, or module should be considered herein as being “configured to” meet that purpose or perform that operation or function.

A controller 600 may be implemented by an algorithm configured to control the operation of various components disposed in a vehicle, a memory configured to store data about a program that reproduces the algorithm, and a processor configured to perform the described operation using data stored in the memory. In this case, the memory and the processor may be implemented as separate chips. Alternatively, the memory and the processor may be implemented as a single chip. For example, the controller 600 may include at least one of an electronic control unit (ECU), a central processing unit (CPU), a microprocessor unit (MPU), a microcontroller unit (MCU), an application processor (AP), or any type of processor well known in the technical field of the present disclosure. Furthermore, the controller 600 may include at least one application configured to execute a method according to the embodiments of the present disclosure, or the same may be formed of a combination of software and hardware capable of performing an arithmetic operation on a program.

Hereinafter, embodiments are described in detail with reference to the accompanying drawings. In describing the embodiments with reference to the accompanying drawings, the same or corresponding components are denoted by the same reference numerals and overlapping descriptions thereof have been omitted.

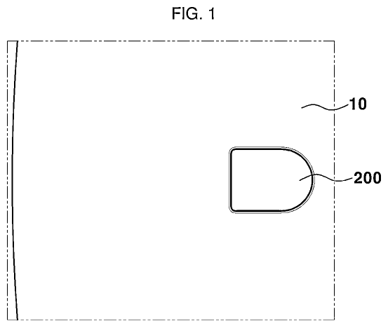

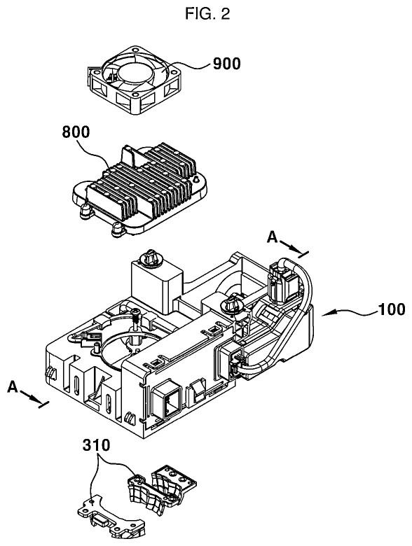

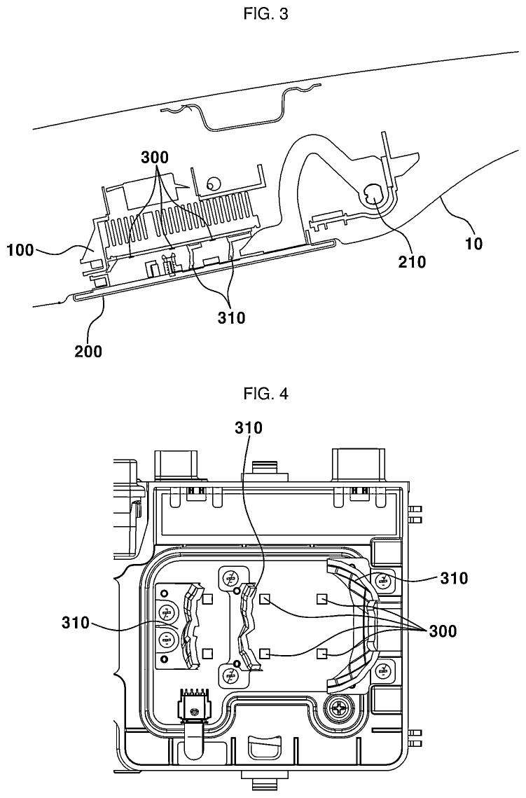

FIG. 1 is a diagram showing one side of a roof lining 10 including a vehicle interior sterilizer of the present disclosure. FIG. 2 is a diagram showing a configuration of the vehicle interior sterilizer. Additionally, FIG. 3 is a side cross-sectional view of an area A-A in FIG. 2.

As shown in the drawings, the vehicle headliner or roof lining 10 is located on the upper surface of the vehicle interior, and the roof lining 10 includes a vehicle interior sterilizer. The vehicle interior sterilizer is housed in a housing 100 inserted into the roof lining 10 so as to be flush with the surface of the roof lining 10. The housing 100 includes an opening therein. A cover unit 200 that is flush with the surface of the roof lining 10 is coupled to the housing 100 and is configured to open and close the opening. The cover unit 200 may be made of or include a surface with the same material as that of the roof lining 10, thereby providing a sense of unity with the roof lining 10.

Moreover, the cover unit 200 is coupled to one end of the housing 100 so as to be rotated around a shaft 210. The cover unit 200 is selectively opened and closed by the shaft 210. One side of the housing 100 facing the cover unit 200 includes the opening and a light source unit 300 is included in the opening.

The light source unit 300 may be formed of at least one light emitting diode (LED), and more particularly may include an ultraviolet LED and a visible LED. As an embodiment of the present disclosure, the light source unit 300 may include a controller 600 configured to select the ultraviolet LED emitting ultraviolet light, the visible light LED emitting visible light, and to control the light emission of each LED.

The ultraviolet LED may emit short-wavelength light in a wavelength range of 10-380 nanometers (nm), or more narrowly, may emit far ultraviolet light in a wavelength range of 200-380 nm. Since the ultraviolet LED plays a role in sterilizing an object, it may be advantageous to emit light in a UV-A region or a UV-C region. In this case, the ultraviolet LED may be manufactured using nitride-based compound semiconductors such as Gallium Nitride (GaN), Aluminum Gallium Nitride (AlGaN), Indium Gallium Nitride (InGaN), or Indium Aluminum Gallium Nitride (InAlGaN).

Additionally, the present disclosure further includes a reflector 310 disposed adjacent to the light source unit 300 and configured to reflect a light source emitted from the light source unit 300. The reflector 310 may be located so as to emit light to a wider area than an interior area directly irradiated with light emitted from the light source unit 300.

The shaft 210 of the cover unit 200 is connected to an actuator unit 400 located in the roof lining 10 adjacent to the housing 100. The driving force of the actuator unit 400 is configured to be applied to one end of the cover unit 200.

According to the embodiment of the disclosure, the actuator unit 400 may include a motor 410 configured to apply a driving force by the controller 600 and a first worm gear 420 located on the central shaft of the motor 410. The actuator unit 400 may also include a second worm gear 440 engaged with a clutch unit 500 circumscribing the shaft 210 and a worm wheel gear 430 located between the first worm gear 420 and the second worm gear 440.

The controller 600 drives the motor 410 to apply rotational force to the clutch unit 500 so that the cover unit 200 is rotated around the shaft 210 and is opened.

In comparison, as another embodiment of the present disclosure, the cover unit 200 is formed of a combination of a plurality of plates 220. The actuator unit 400 is coupled to the back surface of the cover unit 200 to provide the driving force to move the cover unit 200 in one direction so that the cover unit 200 is wound along the outside of the housing 100.

In addition, the sterilizer may include a heat sink 800 located in the housing 100 adjacent to the roof with respect to the light source unit 300 and may include a cooling fan 900 located on one side of the heat sink 800. Accordingly, it is possible to perform a function of dispersing heat generated in the light source unit 300 when the vehicle interior is sterilized by the light source unit 300.

The controller 600 may control the application of power to the actuator unit 400 and may determine whether there is an occupant in the vehicle. Moreover, the controller 600 may receive a sterilization request from a user and may control, in response to the request, the opening of the cover unit 200 and the light emission of the light source unit 300. According to the embodiment of the present disclosure, the controller 600 may determine the presence or absence of an occupant using an interior camera, a seat position sensor, or the like. Upon determining that there is no occupant in the vehicle, the controller 600 is configured to open the cover unit 200 and to allow the light source unit 300 to emit light.

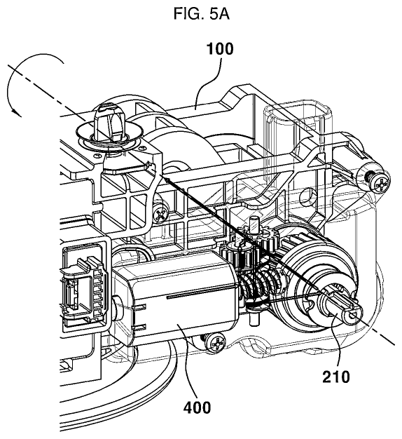

FIG. 4 is a diagram showing a configuration of the light source unit 300 and the reflector 310 located in the housing 100.

As shown in FIG. 4, a plurality of light source units 300 and a plurality of reflectors 310 may be implemented. In one example, six light sources are arranged in two columns and three rows. Additionally, three reflectors 310 are depicted. Two reflectors 310 are located at opposite ends and a third reflector 310 is further located between the first row and the second row of light source units 300. The reflectors 310 may be located so as to disperse light emitted from the light source units 300 over a wider area.

Furthermore, the reflectors 310 are located to minimize uneven energy of light emitted in response to a height of each member disposed in the vehicle. In other words, the reflectors 310 are located to minimize excessive energy of light emitted to a member disposed close to the light source units 300 located in the roof, such as a console or a door handle provided in the vehicle. In addition, as an example of a blind area, light reflected by a reflector 310 is configured to be emitted to a side area of a vehicle seat.

Accordingly, the angle, shape, and position of the reflectors 310 may be freely and selectively adjusted depending on the interior conditions of the vehicle.

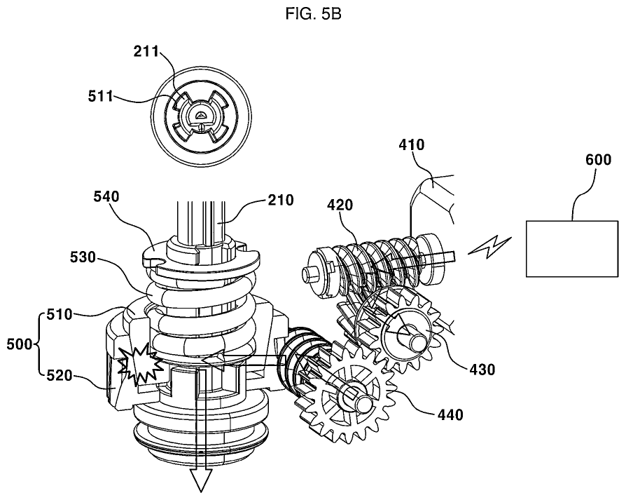

FIGS. 5A and 5B are diagrams showing a coupling relationship between the shaft 210 of the cover unit 200 and the actuator unit 400 according to the embodiment of the present disclosure.

The actuator unit 400 located on the side of the housing 100 or inside the roof lining 10 is located in a state of being coupled to the shaft 210 of the cover unit 200. More particularly, the actuator unit 400 includes the first worm gear 420 located on the central shaft of the motor 410 of the actuator unit 400, the second worm gear 440 engaged with the clutch unit 500 circumscribing the shaft 210, and the worm wheel gear 430 located between the first worm gear 420 and the second worm gear 440.

Upon receiving a request to open the cover unit 200, the controller 600 applies power to the motor 410, and the first worm gear 420 located on the central shaft of the motor 410 is integrally rotated by the applied power. The first worm gear 420 rotated in this manner is configured to apply rotational force to the second worm gear 440 through the worm wheel gear 430. The clutch unit 500 has a clutch gear 520 engaged with the second worm gear 440 and integrally rotated with the shaft 210 by the rotational force of the second worm gear 440.

Furthermore, the clutch gear 520 may be integrally rotated with a clutch plate 510 fixed to the outer peripheral surface of the shaft 210 when rotated in one direction and, when the clutch gear 520 is rotated in the other direction, slip may occur. Accordingly, in response to a request to open the cover unit 200, the clutch gear 520 is engaged with the clutch plate 510 and is integrally rotated with the shaft 210 in the direction of opening the cover unit 200. Additionally, the controller 600 is configured to rotate the motor 410 after sterilization is completed so that the cover unit 200 switches to the closed state. The clutch plate 510 and the clutch gear 520 may be configured to be coupled to each other by frictional force. Therefore, when the clutch gear 520 receives rotational force from the second worm gear 440, the clutch plate 510 and the clutch gear 520 are integrally rotated in one direction of opening the cover unit 200 by frictional force between the clutch plate 510 and the clutch gear 520.

In performing sterilization, when it is determined that failure of a sterilizer has occurred, such as battery discharge or drive device failure, the cover unit 200 does not switch to the closed state thereof. Thereafter, when rotational force is manually applied to the cover unit 200 in the direction of closing the cover unit 200, the clutch plate 510 slips with the clutch gear 520, and the shaft 210 and the clutch plate 510 may be rotated independently of the clutch gear 520.

In other words, when the clutch plate 510 is rotated in the other direction integrally with the shaft 210 through manual operation, an overriding force greater than the frictional force between the clutch plate 510 and the clutch gear 520 is provided so as to rotate the clutch plate 510 independently of the clutch gear 520. Accordingly, the rotational force of the clutch plate 510 may not be transmitted to the clutch gear 520.

In summary, during sterilization, the clutch gear 520 and the clutch plate 510 are integrally rotated in response to rotation in one direction of opening the cover unit 200. After sterilization is completed, the clutch gear 520 and the clutch plate 510 are integrally rotated in the other direction.

When sterilization is completed in a state in which the cover unit 200 is opened due to failure of the sterilizer, or when rotational force is applied in the direction of closing the cover unit 200 by manual operation, the clutch gear 520 and the clutch plate 510 may be moved independently of each other.

Accordingly, in the embodiment of the present disclosure, the actuator unit 400 includes the motor 410. The controller 600 applies power to the motor 410 in response to an opening request or a closing request with respect to the cover unit 200. Conversely, when a user performs a manual operation in the direction of closing the cover unit 200, the clutch plate 510 is rotated in conjunction with the shaft 210 independently of the clutch gear 520 so as to move the cover unit 200 to a closed position thereof.

The clutch plate 510 includes a groove 511 coupled to the shaft 210, and the shaft 210 includes a coupling part 211 inserted into the groove 511. Moreover, the coupling part 211 of the shaft 210 is configured to face one end of the clutch plate 510.

In addition, the vehicle interior sterilizer includes a spring part 530 located between the clutch plate 510 and a stopper 540 located at one end of the shaft 210. The spring part 530 provides elastic force between the stopper 540 located at one end of the shaft 210 and the clutch plate 510 so as to determine the frictional force applied between the clutch plate 510 and the clutch gear 520.

Accordingly, when the cover unit 200 is manually closed, a slipping force between the clutch gear 520 and the clutch plate 510 may be set by the elastic force of the spring part 530.

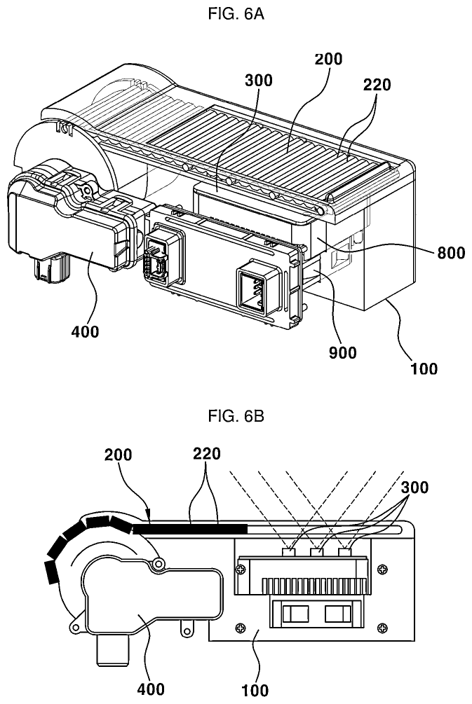

FIGS. 6A and 6B show a sterilizer according to another embodiment of the present disclosure. A plurality of plates 220 are coupled to each other to form the cover unit 200.

In another embodiment, the sterilizer includes the housing 100 having an opening and inserted into the roof lining 10. The sterilizer also includes the light source unit 300 located in the housing 100 and disposed adjacent to the reflector 310.

Additionally, the sterilizer includes the heat sink 800 and the cooling fan 900 sequentially located on the back surface of the light source unit 300. Moreover, the sterilizer includes the actuator unit 400 located on the side surface of the housing 100.

The sterilizer includes a roller (not illustrated) engaged with the actuator unit 400. The roller is coupled to one end of the cover unit 200 formed of a plurality of plates 220. In this case, the plates 220 are configured to be wound along the roller when the actuator unit 400 is driven.

As shown in FIG. 6B, when the actuator unit 400 is driven, the roller engaged with the central shaft of the actuator unit 400 is rotated. The cover unit 200 is wound around the outer peripheral surface of the roller, which is coupled to one end of the cover unit 200 formed of a plurality of plates 220, thereby opening the housing 100.

More particularly, the cover unit 200 is moved in both directions along a slot located in the opening of the housing 100. The housing 100 is controlled to be opened or closed depending on the rotation direction of the roller.

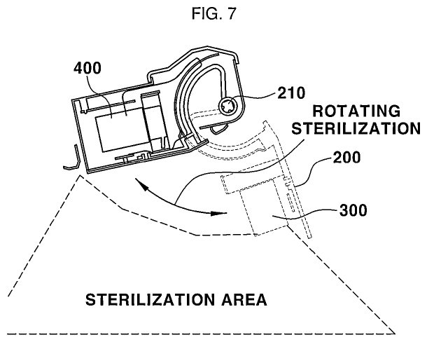

FIG. 7 shows a sterilizer according to still another embodiment of the present disclosure. The light source unit 300 is located on the cover unit 200.

According to still another embodiment of the present disclosure, the sterilizer includes the actuator unit 400 having the motor 410 capable of rotating in both directions. The cover unit 200, which is coupled to one end of the housing 100 and uses the shaft 210, is coupled to the actuator unit 400 and is rotated in both directions so as to open and close the opening of the housing 100.

Moreover, the light source unit 300 is included on the inner surface of the cover unit 200, and the controller 600 is configured to control the light emission of the light source unit 300. Furthermore, since the controller 600 is configured to control the rotation amount of the cover unit 200, an irradiation area irradiated through the light source unit 300 is controlled to be variable.

In other words, the controller 600 is configured to receive a sterilization request from a user and to control the rotation amount of the cover unit 200 in response to the received sterilization request. In this manner, it is possible to vary an area irradiated with light emitted from the light source unit 300 located on the inner side of the cover unit 200.

More particularly, the controller 600 sequentially controls, according to time, the rotation amount of the actuator unit 400 so as to control an opening angle of the cover unit 200. Through this configuration, an area irradiated with light emitted from the light source unit 300 may be controlled to be variable.

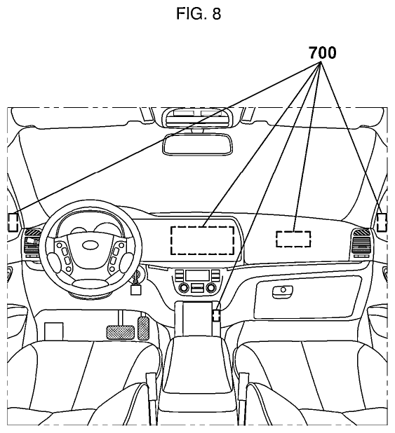

FIG. 8 is a diagram showing an interior structure of a vehicle in which an indicator 700 is located according to the embodiment of the present disclosure.

The indicator 700 of the present disclosure may include a light emitting part attached to and located in the vehicle. Alternatively, a luminescent pigment may be applied to a surface within the vehicle interior so as to emit light by an LED light source.

The indicator 700 formed of the light emitting part is configured to receive power from a vehicle in response to driving of the sterilizer and to allow a user to recognize that sterilization is in progress.

As shown in FIG. 8, the indicator 700 of the present disclosure may be located in or visible in the interior area of the vehicle. One indicator 700 may be disposed in at least one or more of a console, a cockpit module, an instrument cluster monitor, the cover unit 200, a windshield glass, a side glass of the vehicle, or the like.

Further, the indicator 700 formed of a light-emitting pigment may be configured to emit light in response to LED irradiation of the sterilizer without using a power supply. The indicator 700 formed of the light-emitting pigment may be configured to expose a display text when the sterilizer is driven.

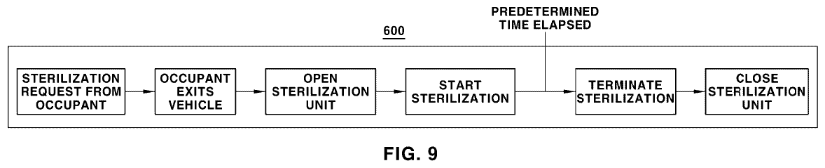

FIG. 9 is a diagram showing control steps of operating the sterilizer through the controller 600.

The controller 600 of the present disclosure determines the presence or absence of an occupant before the sterilizer is driven. In order to prevent an occupant from being exposed to ultraviolet rays during the operation or driving of the sterilizer, the sterilizer is driven based on the condition that there is no occupant in the vehicle. More particularly, the controller 600 determines whether an occupant is present in the vehicle based on conditions indicating that an occupant exits the vehicle, such as an opened state of the door, interior camera photographing information, or a seat position sensor.

When the controller 600 determines that there is no occupant in the vehicle and the sterilization request is approved, the cover unit 200 is opened and then power is applied to the light source unit 300, thereby performing sterilization for a predetermined time.

When sterilization is normally completed, the controller 600 applies power to the actuator unit 400 so as to close the cover unit 200. However, when breakdown or failure of the sterilizer occurs during the sterilization step, the controller 600 is configured to terminate sterilization in a state in which the cover unit 200 is opened.

As should be apparent from the above description, the present disclosure may achieve the following effects through the embodiments, a combination of the above-described configurations, and a use relationship therebetween.

The present disclosure has an effect of maintaining a vehicle in a sterilized state by providing a vehicle interior sterilizer capable of sterilizing contaminated areas of the vehicle interior surfaces.

In addition, the present disclosure has an effect of preventing deterioration in the interior design of a vehicle by providing a vehicle interior sterilizer that is not exposed to the outside, i.e., exposed through the roof liner.

Furthermore, the present disclosure is configured to perform sterilization after determining whether an occupant is present in the vehicle, thereby making it possible to provide a vehicle interior sterilizer capable of ensuring the safety of the occupant.

The present disclosure has been described in detail with reference to various embodiments thereof. However, the present disclosure may be used in various other combinations, modifications, and environments. In other words, it should be appreciated by those having ordinary skill in the art that changes may be made in these embodiments without departing from the principles and spirit of the disclosure, the scope of which is defined in the appended claims and equivalents thereto. The embodiments describe implementing the technical idea of the present disclosure.

Various changes required in specific application fields and uses of the present disclosure are also possible. Accordingly, the detailed description of the present disclosure is not intended to limit the present disclosure to the disclosed embodiments. Additionally, the scope of the appended claims should be construed as including other embodiments as well.

Reference numerals set forth in the Drawings include reference to the following elements as further discussed below:
10: Roof lining 100: Housing
200: Cover unit 210: Shaft
211: Coupling part 220: Plate
300: Light source unit 310: Reflection part
400: Actuator unit 410: Motor
420: First worm gear 430: Worm wheel gear
440: Second worm gear 500: Clutch unit
511: Clutch plate 510: Groove
520: Clutch gear 530: Spring part
540: Stopper 600: Controller
700: Indicator 800: Heat sink
900: Cooling fan

What is claimed is:

1. A vehicle interior sterilizer comprising:
a roof lining;
a housing inserted into and located in the roof lining, the housing having an opening;
a cover unit configured to open and close the opening of the housing;
an actuator unit coupled to a shaft of the cover unit and configured to apply a driving force to selectively open and close the cover unit;
a light source unit located in the opening of the housing and exposed to an interior of a vehicle so as to perform sterilization when the cover unit is opened; and
a controller configured to determine whether an occupant exits the vehicle and to open the cover unit and turn on the light source unit by driving of the actuator unit.

2. The vehicle interior sterilizer of claim 1, further comprising a reflector located adjacent to the light source unit.

3. The vehicle interior sterilizer of claim 1, further comprising an indicator located in at least one or more of the cover unit, a cockpit module, a console, an instrument cluster monitor, a windshield, or a vehicle side glass,
wherein the indicator is configured to light up when the light source unit is driven.

4. The vehicle interior sterilizer of claim 3, wherein the indicator includes a luminescent pigment applied thereto.

5. The vehicle interior sterilizer of claim 1, wherein the actuator unit comprises:
a motor configured to provide a driving force;
a first worm gear engaged with a central shaft of the motor;
a worm wheel gear engaged with the first worm gear; and
a second worm gear located between the worm wheel gear and a clutch unit circumscribing the shaft.

6. The vehicle interior sterilizer of claim 5, wherein the clutch unit comprises:
a clutch plate integrally rotated with the shaft; and
a clutch gear configured to surround an outer surface of the clutch plate and engage with the second worm gear.

7. The vehicle interior sterilizer of claim 6, further comprising:
a spring part configured to surround an outer peripheral surface of the shaft, wherein the spring part is configured to press the clutch plate so that the clutch plate and the shaft are integrally secured; and
a stopper disposed to face an end of the spring part and configured to regulate a position of the spring part.

8. The vehicle interior sterilizer of claim 6, further comprising:
a groove formed in the clutch plate; and
a coupling part located at one end of the shaft,
wherein the one end faces the clutch plate is and inserted into the groove.

9. The vehicle interior sterilizer of claim 6, wherein, when the cover unit is rotated in a direction of closing the cover unit in response to a manual operation, the clutch plate is configured to prevent rotational force of the cover unit from being applied to the clutch gear.

10. The vehicle interior sterilizer of claim 1, wherein:
the cover unit is formed of a plurality of plates; and
the plurality of plates is configured to move to a side surface of the housing in response to the driving of the actuator unit so as to open the opening of the housing.

11. The vehicle interior sterilizer of claim 1, wherein, when a sterilization request from a user is approved, the controller is configured to apply power to the actuator unit and to switch the cover unit to an opened state thereof.

12. The vehicle interior sterilizer of claim 1, further comprising:
a heat sink located on one side of the light source unit; and
a cooling fan located on one side of the heat sink.

13. A vehicle interior sterilizer comprising:
a roof lining;
a housing inserted into and located in the roof lining, the housing having an opening;
a cover unit configured to open and close the opening of the housing;
an actuator unit coupled to a shaft of the cover unit and configured to apply a driving force so as to open and close the cover unit;
a light source unit located in the cover unit and exposed to an interior of a vehicle so as to perform sterilization when the cover unit is opened; and
a controller configured to determine whether an occupant exits the vehicle and to open the cover unit and turn on the light source unit by driving of the actuator unit.

14. The vehicle interior sterilizer of claim 13, wherein, when switching the light source unit to an on state thereof, the controller is configured to control rotational force of the actuator unit so that the cover unit is rotated around the shaft by a predetermined angle.

15. The vehicle interior sterilizer of claim 13, further comprising an indicator located in at least one or more of the cover unit, a cockpit module, a console, an instrument cluster monitor, a windshield, or a vehicle side glass,
wherein the indicator is configured to light up when the light source unit is driven.
